(19) United States
(12) Patent Application Publication
(10) Pub. No.: US 2009/0142735 A1
(43) Pub. Date: Jun. 4, 2009

(54) Bacterial intraoral disease treatment composition, washing treatment solution, hemostatic treatment solution, and bacterial intraoral disease treatment method

(76) Inventors: Toyohiko Takushige, Miyagi (JP); Etsuro Hoshino, Niigata (JP)

Correspondence Address: KNOBBE MARTENS OLSON & BEAR LLP, 2040 MAIN STREET, FOURTEENTH FLOOR, IRVINE, CA, 92614, US

(21) Appl. No.: 10/583,500

(22) PCT Filed: Mar. 10, 2006
(86) PCT No.: PCT/JP06/04762

§ 371 Date: Jun. 19, 2006

(30) Foreign Application Priority Data
Mar. 11, 2005 (JP) 2005-070168

(51) Int. Cl. A61C 19/06; A61K 8/72; A61K 8/27; A61Q 11/00

(52) U.S. Cl. 433/224; 424/49

(57) ABSTRACT

A treatment method is provided that is entirely different from conventional treatment methods in that it is capable of sufficiently suppressing pain and the recurrence of all the bacterial intraoral diseases without depending on removal of living tissues. This treatment method is a lesion-sterilizing and tissue-repairing treatment method including the processes of: washing teeth affected with bacterial intraoral disease with a washing treatment solution; administering a treatment composition to the washed teeth; and covering an opening of the teeth. This treatment composition includes an antibacterial agent having an antibacterial property against intraoral bacteria and a base containing polyethylene glycol 400, polyethylene glycol 600, polyethylene glycol 6000, and propylene glycol.

### TECHNICAL FIELD

The present invention relates to, for example, a bacterial intraoral disease treatment composition, a washing treatment solution, a hemostatic treatment solution, and a bacterial intraoral disease treatment method.

### BACKGROUND ART

Conventionally, as a typical example of a treatment method for bacterial intraoral diseases (such as caries, pulp disease, apical periodontal disease and periodentitis), a treatment method provided with the following constitution has been cited.

FIG. 8 depicts a cross-sectional view of a tooth 100 affected with caries, an example of a bacterial intraoral diseases, and pulp disease.

The tooth 100 is of a structure comprising, from the outside, in order, enamel 130, dentin 140, and dental pulp 150, and embedded into the alveolar bone 180 via cement 160 and a periodontal membrane 170. This alveolar bone is covered with gingival 190.

The invasion of the intraoral bacteria 111 into the inside of the tooth 100 results in the formation of a dental caries site 110. This dental caries site 110 comprises free enamel, a smear layer, tissue in which bacteria exist, etc.

FIG. 9 is a cross-sectional view showing the state of the tooth 100′ when the tooth 100 of FIG. 8 has been treated with a treatment method according to the conventional example.

The above-described conventional treatment method comprises the processes of: grinding off the caries site 110 from the tooth 100 affected with the bacterial intraoral disease to remove the dental pulp 150, and covering the opening 120′ of the tooth 100′ with a filling material (not shown) from which the caries site 100 has been ground off and the dental pulp 150′ has been removed (see, e.g., Patent Document 1).

With this treatment method, since the opening 120′ of the tooth 100′ is covered after the dental caries site 110 is ground off and the dental pulp 150 is removed, the bacterial intraoral disease can be treated as far as the intraoral bacteria 111 are completely eliminated.

Patent Document 1 Japanese Laid-Open Patent Application Publication No. 2002-541907

### DISCLOSURE OF THE INVENTION

However, with the above-described treatment method, when the invasion of intraoral bacteria 111 has reached the dental pulp 150, in order to eliminate the intraoral bacteria 111 and avoid causing toothache after the treatment, there is no other way but to remove the dental pulp 150 of the crown, generally the dental pulp 150 not only of the crown but also from the crown to the root of tooth (see FIG. 9).

Also due to the extreme difficulty in accurately identifying the range of the caries site 110, the complete elimination thereof is often hindered in the grinding process. When the caries site 110 cannot be completely eliminated, problems are posed such as the recurrence of bacterial intraoral disease due to the repropagation of the remaining intraoral bacteria inside the tooth 100′ after the aforementioned covering process.

Accordingly, in order to improve the possibility of complete elimination of the caries site 110, measures have been taken to grind off not only the site identified as the caries site 110 but also the surrounding sites thereof.

However, with the progress of the disease, when the intraoral bacteria 111 spread to the depth of the dentin 140, the cement 160, and the periodontal membrane 170, the complete elimination of the intraoral bacteria 111 becomes more difficult.

Accordingly, in such a case, there is no other way but to abandon the treatment by grinding and deal with the intraoral disease by the extraction of the tooth 100 as a whole.

Thus the conventional treatment method depended on the exhaustive elimination of living tissue to prevent the recurrence of bacterial intraoral disease.

The present invention has been made in view of the above-described problems, and one object thereof is to provide a treatment method independent of the elimination of living tissue and capable of sufficiently suppressing pain caused to the subject and the recurrence of bacterial intraoral diseases, a treatment composition, a washing treatment solution, and a hemostatic treatment solution.

The inventors of the present invention have adopted a treatment method of internal medicine, which is entirely different from the conventional treatment method depending on the physical elimination of living tissue, to the dental treatment, thereby achieving the present invention.

More specifically, the present invention provides the following.

(1) A treatment method for the bacterial intraoral diseases comprising the processes of: washing a tooth affected by bacterial intraoral disease with a washing treatment solution, administering a treatment composition to the washed tooth, and covering the tooth opening, in which the treatment composition comprises:

an antibacterial agent having an antibacterial property against intraoral bacteria and

a base containing polyethylene glycol 400, polyethylene glycol 600, polyethylene glycol 6000 and propylene glycol.

Here, “bacterial intraoral disease” refers to intraoral diseases caused by bacterial infections at all stages thereof including, but not limited to, caries, pulp disease, apical periodontal disease, and periodontitis.

(2) The treatment method according to (1) in which the above-described base contains polyethylene glycol 400 of not less than 13 percent volume and not more than 19 percent volume of this base, polyethylene glycol 600 of not less than 13 percent volume and not more than 19 percent volume, polyethylene glycol 6000 by not less than 27 percent volume and not more than 38 percent volume, and propylene glycol by not less than 36 percent volume and not more than 50 percent volume.

(3) The treatment method according to (1) or (2) in which the above-described washing treatment solution contains EDTA at a pH of about 7 and a water-soluble thickener containing no metallic ions.

(4) The treatment method according to (3) in which the above-described washing treatment solution contains EDTA of not less than 10 percent volume and not more than 12 percent volume of this washing treatment solution.

(5) The treatment method according to any one of (1) to (4) which further comprises, prior to the above-described covering process, a hemostatic process of stopping the bleeding from the dental pulp and/or gingival in which the above-described hemostatic process is a process of stopping bleeding from the dental pulp and/or gingival using a hemostatic treatment solution containing sodium alginate and zinc oxide.

(6) The treatment method according to (5) in which the above-described hemostatic treatment solution contains sodium alginate of not less than 6.0 percent volume and not more than 6.5 percent volume—and zinc oxide of not less than 33 percent volume and not more than 35 percent volume of this hemostatic treatment solution.

(7) A treatment composition for bacterial intraoral disease which has an antibacterial agent having an antibacterial property against intraoral bacteria and a base containing polyethylene glycol 400, polyethylene glycol 600, polyethylene glycol 6000, and propylene glycol.

(8) The treatment composition according to (7) in which the above-described base contains polyethylene glycol 400 of not less than 13 percent volume and not more than 19 percent volume of this base, polyethylene glycol 600 of not less than 13 percent volume and not more than 19 percent volume, polyethylene glycol 6000 of not less than 27 percent volume and not more than 38 percent volume, and propylene glycol of not less than 36 percent volume and not more than 50 percent volume.

(9) A washing treatment solution to be used for washing teeth which contains EDTA at a pH of about 7 and a water-soluble thickener containing no metallic ions.

(10) The washing treatment solution according to (9) which contains EDTA of not less than 10 percent volume and not more than 12 percent volume.

(11) A hemostatic treatment solution to be used to stop bleeding from dental pulp and/or gingival which contains sodium alginate and zinc oxide.

(12) The hemostatic treatment solution according to (11) which contains sodium alginate of not less than 6.0 percent volume and not more than 6.5 percent volume and zinc oxide of not less than 33 percent volume and not more than 35 percent volume.

(13) A treatment kit for bacterial intraoral diseases which comprises basal ingredients for the treatment composition according to (7) or (8), the washing treatment solution according to (9) or (10), and the hemostatic treatment solution according to (11) or (12).

“Basal ingredients” refer to the respective compounds constituting the treatment composition in the state prior to being mixed together. More specifically, the respective compounds are antibacterial agents including metronidazole, minocycline, and ciprofloxacin as well as a base including polyethylene glycol 400, polyethylene glycol 600, polyethylene glycol 6000, and propylene glycol.

### BRIEF DESCRIPTION OF THE DRAWINGS

FIG. 1 depicts a cross-sectional view of a tooth affected with bacterial intraoral disease at an early stage of treatment by a treatment method according to a first embodiment of the present invention.

FIG. 2 depicts a cross-sectional view of the tooth of FIG. 1 at a subsequent stage, at which the tooth has been treated by the treatment method according to the above-described embodiment.

FIG. 3 depicts a cross-sectional view of the tooth of FIG. 1 at a further subsequent stage, at which the tooth has been treated by the treatment method according to the above-described embodiment.

FIG. 4 depicts a cross-sectional view of a tooth affected with bacterial intraoral disease at an early stage of treatment by the treatment method according to a second embodiment of the present invention.

FIG. 5 depicts a cross-sectional view of the tooth of FIG. 4 at a subsequent stage, at which the tooth has been treated by the treatment method according to the above-described embodiment.

FIG. 6 depicts a cross-sectional view of the tooth of FIG. 4 at a further subsequent stage, at which the tooth has been treated by the treatment method according to the above-described embodiment.

FIG. 7 depicts a cross-sectional view of a sample in a test example of the present invention.

FIG. 8 depicts a cross-sectional view of a tooth affected with bacterial intraoral disease.

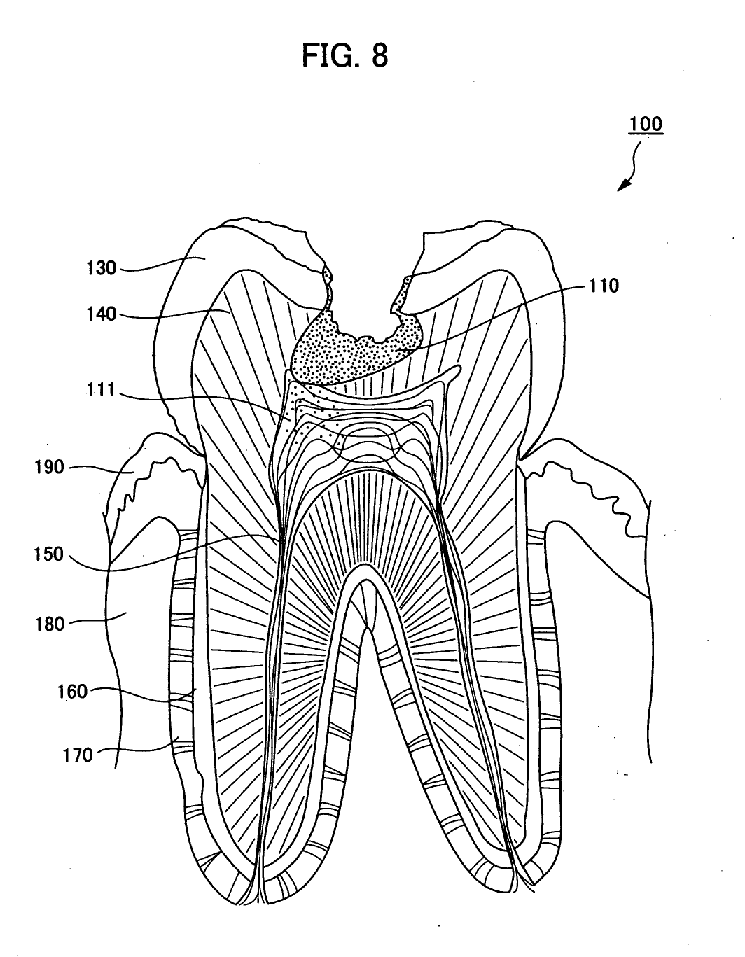

FIG. 9 depicts a cross-sectional view of the tooth of FIG. 8 which has been treated by a treatment method according to a conventional example.

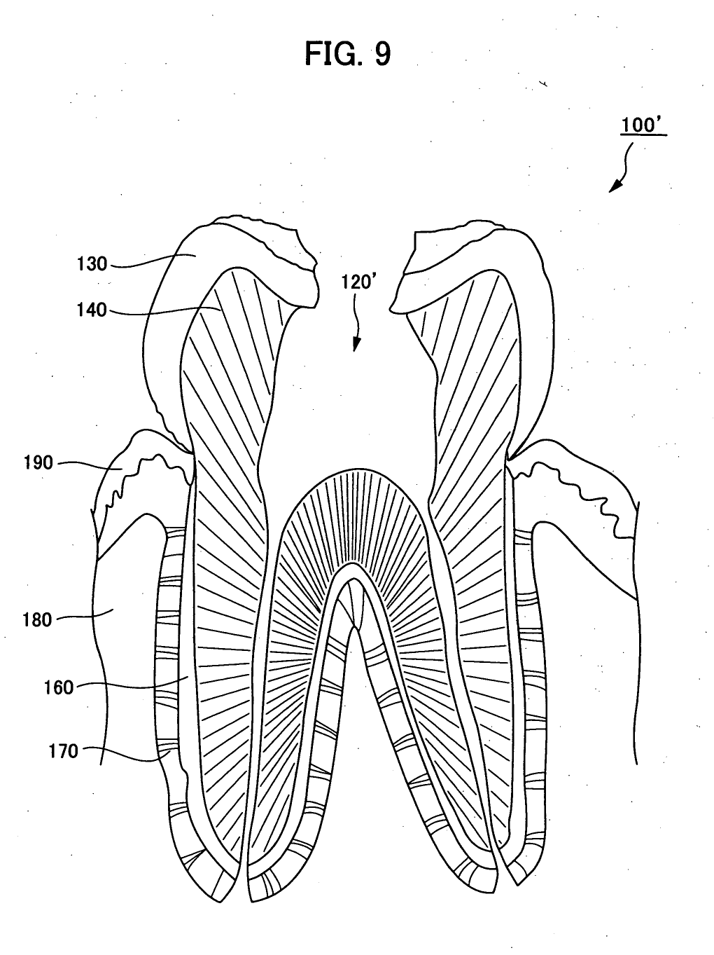

### DESCRIPTION OF SYMBOLS AND NUMERALS

1 tooth,

13 treatment drug layer

14 latching site

16 intraoral bacteria

21 filling material layer

22 glass-ionomer cement layer

23 dental luting agent layer

24 sterilized tissue

### PREFERRED MODE FOR CARRYING OUT THE INVENTION

In the following, preferred embodiments of the present invention are described with reference to the drawings. Herein, in descriptions of respective embodiments other than the first embodiment, items common to those in the first embodiment are marked with the same symbol (numeral), and descriptions thereof are omitted or simplified.

### First Embodiment

### Treatment Composition

Treatment composition of the present invention has an antibacterial agent and a base.

### Antibacterial Agent

Antibacterial agent has an antibacterial property against intraoral bacteria. This antibacterial agent is a combination of desired antibiotics for killing all of a wide variety of intraoral bacteria, including, for example, metronidazole, minocycline, and ciprofloxacin. A combination of these three types of ingredients is considered to exert the antibacterial action against all of the intraoral bacteria.

Considering antibacterial effects against intraoral bacteria, content ratios of the respective ingredients are preferably set such that potency ratios of metronidazole:minocycline:ciprofloxacin are 1˜3:1:1.

There are no particular limitations to metronidazole, and, for example, “Asuzole tablet 250 mg (trade name)” (Fuji Pharmaceutical Co. Ltd.) may be used.

There are no particular limitations to minocycline, and, for example, “Minomycine 100 mg (trade name)” (Weis Inc.) may be used.

There are no particular limitations to ciprofloxacin, and, for example, “Cyproxane 200 mg (trade name)” (Bayer AG) may be used.

When these commercial products are used, drug ingredients gotten rid of drug coating materials and capsules may be used.

### Base

The base stabilizes treatment effects by antibacterial agents on bacterial intraoral disease, specifically including polyethylene glycol and propylene glycol.

Polyethylene glycol is mixed with the powdered antibacterial agents to change them into a pasty or ointment-like preparation, thereby improving the operability thereof and facilitating the measurement of doses of the treatment composition.

A polyethylene glycol base containing polyethylene glycol 400, polyethylene glycol 600, and polyethylene glycol 6000 may be used.

Polyethylene glycol 400 improves penetrability of the treatment composition. Polyethylene glycol 600 has a melting point of about 18 degrees Celsius such that it is solid extraorally for easy loading thereof onto administering tools, while it is liquidized intraorally and improved in its soaking properties for its administering easily to teeth. In addition, polyethylene glycol 6000 increases viscosity of the treatment composition thereby improving the operability.

Besides these, a mixture of polyethylene glycol 4000 and polyethylene glycol 400 can be also used.

In addition, there are no particular limitations to polyethylene glycol, and, for example, “Solbase (brand name)” (Dainippon Pharmaceutical Co.) may be used.

Propylene glycol adjusts the viscosity of anti-bacterial agents which have been changed into pasty or ointment-like preparations. Adjustment of the treatment composition to the desired viscosity results in improving the penetrability thereof. Propylene glycol also has sterilizing effects against intraoral fungi.

Considering the effects of various ingredients as described above, the base preferably contains polyethylene glycol 400 at not less than 13 percent volume and not more than 19 percent volume, polyethylene glycol 600 at not less than 13 percent volume and not more than 19 percent volume, polyethylene glycol 6000 at not less than 27 percent volume and not more than 38 volume percent, and propylene glycol at not less than 36 percent volume and not more than 50 percent volume.

### Preparation Method

First, the desired amounts of antibacterial agents are separately placed into mortars and pulverized with a pestle to prepare the anti-bacterial agents for mixing.

Furthermore, after a desired amount of propylene glycol is poured into a beaker-shaped vessel, polyethylene glycol is added in small portions to this propylene glycol until the desired viscosity is reached, and gently mixed to obtain the base.

These antibacterial agents and the base are mixed in the desired ratios to prepare the treatment composition.

Considering that there is a possibility that when the base content is too high, the treatment composition is so soft that the operability thereof conversely deteriorates and at the same time may become unable to thoroughly sterilize bacteria due to insufficient antibacterial agents, antibacterial agents may be mixed so as to contained not less than 5 percent volume and not more than 7 percent volume (volume ratio) with respect to the base in general.

In this case, the treatment composition readily deteriorates to lose treatment effects thereof. Thus, it is preferably prepared just prior to its use. In other words, basal ingredients of the treatment composition, antibacterial agents in particular, are preferably stored separately in a dark cold place. Furthermore, when the treatment composition is stored after its preparation, in order to prevent its deterioration, it is preferably stored in a sealed vessel under shaded conditions with low temperature and low humidity. However, even under such conditions, period during which the treatment composition is effective is usually about two days.

### Washing Treatment Solution

The washing treatment solution of the present invention is used for washing teeth, specifically containing EDTA at a pH of about 7 and a water-soluble thickener without metallic ions.

EDTA acts as a chelating agent for the free calcium of teeth. Accordingly, the washing treatment solution containing EDTA chelates and eliminates the free calcium so as to remove microorganisms embedded in the free calcium. Furthermore, since the washing treatment solution, if acidic, decalcifies teeth, and, if alkaline, inhibits the calcium precipitation, the pH of the washing treatment solution is preferably near 7.

In addition, when the EDTA content is too low, the efficiency of capturing the free calcium becomes insufficient, while, when too high, the efficiency of capturing the free calcium is saturated so as to be disadvantageous from the point of view of cost. Therefore, the EDTA content is preferably not less than 10 percent volume and not more than 12 percent volume.

Thickeners confer viscosity on the washing treatment solution to retard the outflow thereof from teeth and secure the time required for performing elimination of microorganisms and such by EDTA. Furthermore, since thickeners preferably contain no metallic ions in order that thickeners are stable with respect to EDTA. An example of such thickeners is dextrin.

When the dextrin content is too small, the washing treatment solution rapidly flows out from the teeth due to the insufficient viscosity thereof, while when too large, the penetration of the EDTA into the teeth is inhibited. The dextrin content is preferably not less than 2.7 percent volume and not more than 3.0 percent volume.

Although solvents for the washing treatment solution are not particularly limited, one example thereof is purified water.

### Preparation Method

Dotite 2NA and Dotite 4NA are added in this order to the purified water in equal amounts (by volume ratio) to dissolve them. After the dissolution, the amount of the purified water is adjusted such that the EDTA concentration becomes 24 percent volume.

To the EDTA concentration-adjusted solution is added an equal amount (by volume ratio) of dextrin, and the solution is mixed to prepare the washing treatment solution. As a result, the washing treatment solution contains EDTA at a concentration of 12 percent volume.

### Hemostatic Treatment Solution

The hemostatic treatment solution of the present invention is used to stop bleeding from dental pulp and gingival when they bleed in a removal process, washing process, etc., described below. Specifically, the hemostatic treatment solution contains sodium alginate and zinc oxide.

Sodium alginate covers the bleeding site to suppress historrhexis from mucosa.

Zinc oxide binds with proteins existing in teeth and gingival to form a film so as to exert vasoconstrictive, antiphlogistic, protective, and antiseptic actions. Zinc oxide also absorbs exudate and suppresses the secretion thereof to dry the wounded surface.

When the sodium alginate content is too low, it cannot sufficiently suppress the historrhexis from mucosa, while when too high, the hemostatic treatment solution does not diffuse well over the entire bleeding site due to the excessive increase in its viscosity. The sodium alginate content is preferably not less than 6.0 percent volume and not more than 6.5 percent volume.

When the zinc oxide content is too low, it cannot sufficiently stop the bleeding from a wounded surface, while when too high, the above-described action is saturated so as to be disadvantageous in the aspect of cost. The zinc oxide content is preferably not less than 33 percent volume and not more than 35 percent volume.

There are no particular limitations to solvents for the hemostatic treatment solution, and, for example, the purified water may be cited.

### Preparation Method

Sodium alginate is added to the purified water at a 10:1 volume ratio for dissolution. To the solution thus obtained, zinc oxide is added at a 100:55 volume ratio for dissolution to prepare the hemostatic treatment solution.

In the following, one embodiment of the treatment method of the present invention is explained with reference to the figures.

### Treatment Method

A treatment method in the present invention includes the processes of: washing teeth affected with bacterial intraoral disease using the washing treatment solution; administering the treatment composition to the washed teeth, and covering the teeth opening.

The treatment method may further include, prior to the washing process, the processes of: forming a latching site to which the covering material is latched in the covering process, and forming an administrating site on which a sufficient amount of the treatment composition is loaded. The treatment method may further include, prior to the covering process, a hemostatic process to stop the bleeding from the dental pulp.

### Latching Site Formation Process

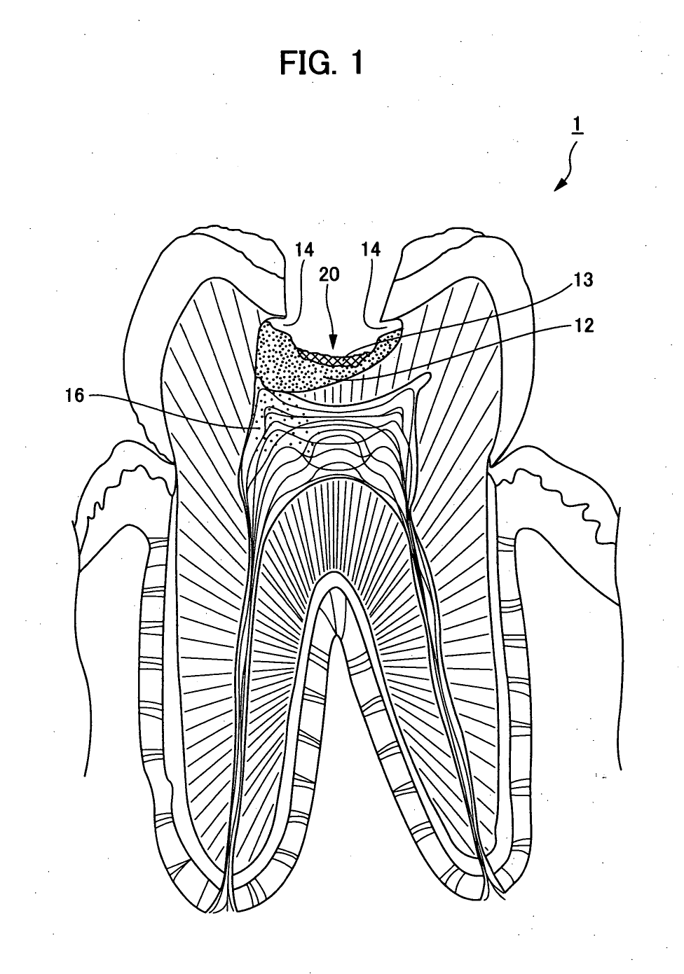

The latching site formation process is a process of mechanically removing the free calcium (e.g. free enamel and smear layer) from a tooth affected with bacterial intraoral disease to form a latching site to which covering materials are latched (the latching sites 14 in the FIGS. 1 to 3 described below). The latching site formation may be performed using the known means (such as an excavator and turbine bar).

From the aspect of suppressing pain to the subject, it is preferable to avoid grinding not only living tissues but also necrotic tissues (softened dentin in particular) as much as possible. In this case, although nerves in necrotic tissue are dead so that the removal thereof primarily causes no pain to a subject, the operation often stimulates nerves near the necrotic tissue to cause pain to the subject.

### Administering Site Formation Process

The administering site formation process is a process of forming a sufficiently wide administering site in the following administering process when a sufficient space for loading the treatment composition is not present in the subject's tooth, or a process to promote the treatment composition delivery to the bacterial invasion site of alveolar bone and the like, in the case of treatment of an infected root canal. That is, this administering site formation process is an optional process to be arbitrarily performed, taking the bacterial invasion range and such into consideration.

In the case of an intraoral bacteria infection which has reached the root canal and dental pulp, even when it is difficult to widen and build the root canal and even when the root of the tooth is curved, a mere loading of the treatment composition on the opening of the root canal enables the passage of this treatment composition through the dentinal canals and gaps between the root canal and root-filling material as well as the diffusion and penetration thereof into the root canal and dental pulp.

### Hemostatic Process

The hemostatic process is a process of stopping the bleeding from dental pulp and gingival prior to the washing process described below. Hemostasis may be performed using the above-described hemostatic treatment solution, and, more specifically, after covering the bleeding site with this hemostatic treatment solution usually for about 1 to 2 minutes, it is removed by gently applying a water gun to the treated site.

Since the remaining hemostatic treatment solution interferes with treatment effects of the treatment composition administered in the administration process described below, it is preferable to remove the hemostatic treatment solution as much as possible.

### Washing Process

The washing process is a process of washing teeth after the latching site formation process and before the administering process described below. Washing of the remaining free calcium in the washing process further promotes the following diffusion and penetration of the treatment composition into bacteria-infected tissues.

Washing may be performed using the above-described washing treatment solution, specifically by spouting the washing treatment solution from the tip of a washing tool provided with a fine tube nozzle.

### Administration Process

The administration process is a process of administering the above-described treatment composition to a tooth from which the free calcium has been removed. More specifically, a nearly spherical treatment composition of about 1 mm in diameter is loaded on a suitable position of tissue in which bacteria exist. Thereby the loaded treatment composition diffuses and penetrates into tissue with bacteria so as to sterilize the bacteria-invaded site.

FIG. 1 depicts a cross-sectional view of the tooth 1 in an early stage of treatment by the treatment method according to a first embodiment of the present invention.

In the tooth 1, the caries site 12 is formed in the dentin due to the invasion of intraoral bacteria, which have reached the dental pulp as shown by dots in FIG. 1. In this tooth 1, the treatment composition is layered on the administering site built up on the caries site 12 to form the treatment drug layer 13.

Other structures of the tooth l are common to those of the above-described tooth 100 and explanations thereof are omitted.

### Covering Process

The covering process is a process of covering the opening of a tooth administered with the treatment composition. By covering the opening, the invasion of intraoral bacteria into the sterilized site is blocked so as to maintain sterile conditions. Specifically, the tooth opening is covered with a filling material (e.g. glass-ionomer cement “Fuji IX GP (brand name)” (G C Co., Ltd.)) so as to cover the administered treatment composition.

In addition, in the case of root canal treatment, after the above-described treatment composition is applied to its administering seat, it may be covered with hydraulic cement (e.g. “Caviton (registered trade mark)” (G C Co., Ltd.)) which may be further covered with phosphate cement.

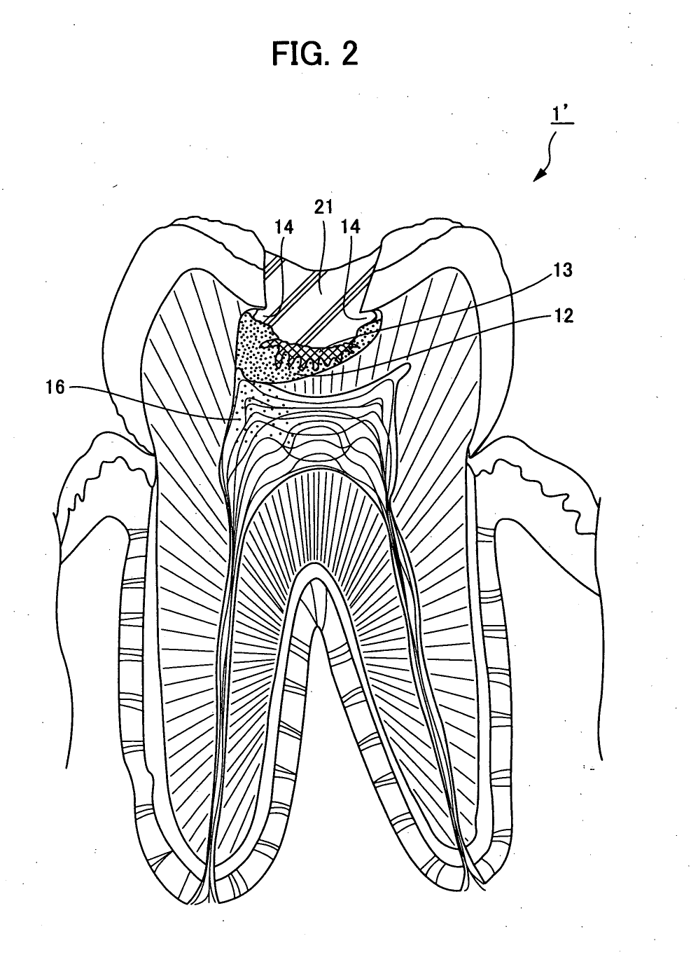

FIG. 2 depicts a cross-sectional view of the tooth 1′ in the next stage of treating the tooth 1 of FIG. 1 by the treatment method according to the first embodiment of the present invention.

In the tooth 1′, filling materials are layered over the above-described opening 20 to build up the filling material layer 21, which is latched to the tooth 1′ by the latching sites 14. Other structures of the tooth 1′ are common to those of the above-described tooth 100 and the explanations thereof are omitted.

When left standing under these conditions, the treatment composition diffuses from the treatment drug layer 13 to the caries site 12 and sterilizes the caries site 12 with this diffusion to form sterilized tissue 24.

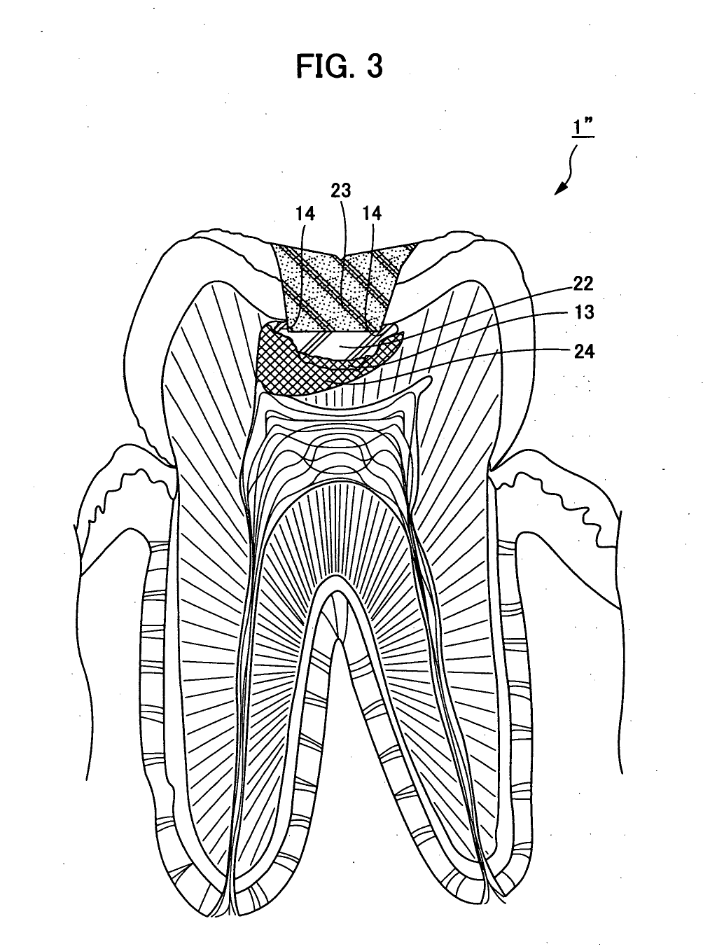

FIG. 3 depicts a cross-sectional view of the tooth 1″ in a further stage of treating the tooth 1 of FIG. 1 by the treatment method according to the first embodiment of the present invention.

In the tooth 1″, the glass-ionomer cement (“Fuji IX GP (brand name)”) and the luting agent in which crown-repairing element is bound with adhesive resinous cement are layered in this order over the opening 20 which has been exposed by removing the above-described filling material layer 21, thereby forming the glass-ionomer cement layer 22 and the luting agent layer 23. Furthermore, the treatment composition diffuses into the dental pulp so as to eliminate the intraoral bacteria 16.

Other structures of the tooth 1″ are common to those of the above-described tooth 100 and explanations thereof are omitted.

With the treatment method according to the above-described embodiment, formation of the treatment drug layer 13 over the caries site 12 alone enables the diffusion of the treatment composition over the entire tooth 1 and sterilization of intraoral bacteria. Accordingly, even though intraoral bacteria have reached the depth of dentin, bacterial intraoral diseases can be treated without removing living tissue.

### Second Embodiment

This embodiment differs from the first embodiment in the treatment method constitution.

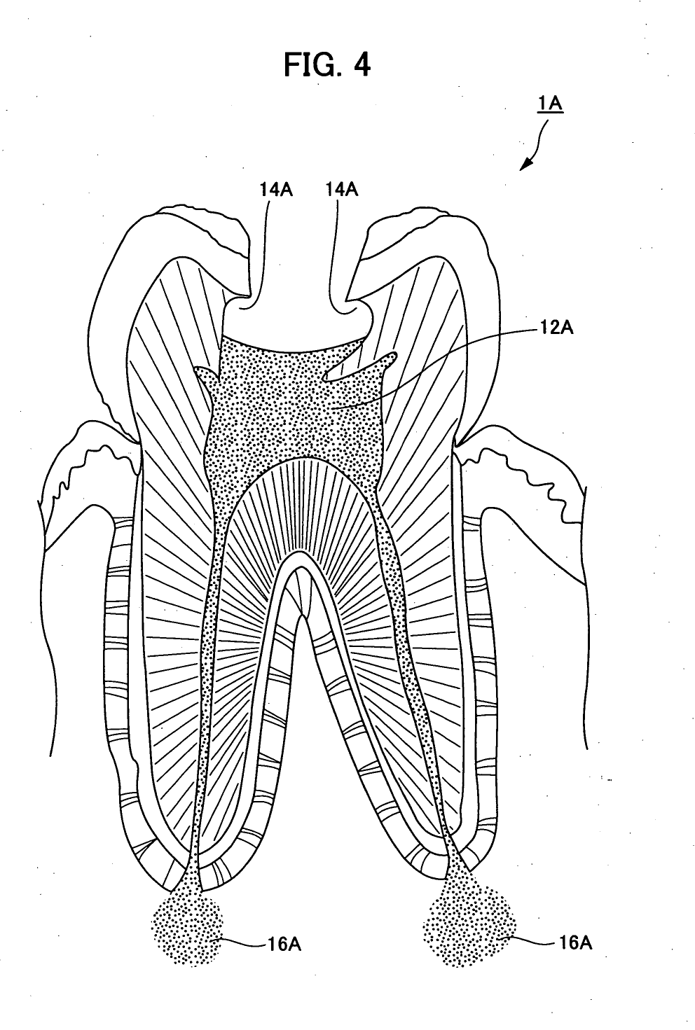

FIG. 4 depicts a cross-sectional view of the tooth 1A in an early stage of treatment by the treatment method according to the second embodiment of the present invention.

In the tooth 1A, the intraoral bacteria 16A necrotize the dental pulp further invading the alveolar bone from the inside of the tooth. When the tooth is left untreated under such conditions, periodontitis is induced.

A treatment method in the present invention for such symptoms (infected root canal treatment) is further provided with a root canal filling process in which necrotic pulps within the root canal are removed and this root canal is filled with the filling material mediating diffusion of the treatment composition.

### Administration Site Formation Process

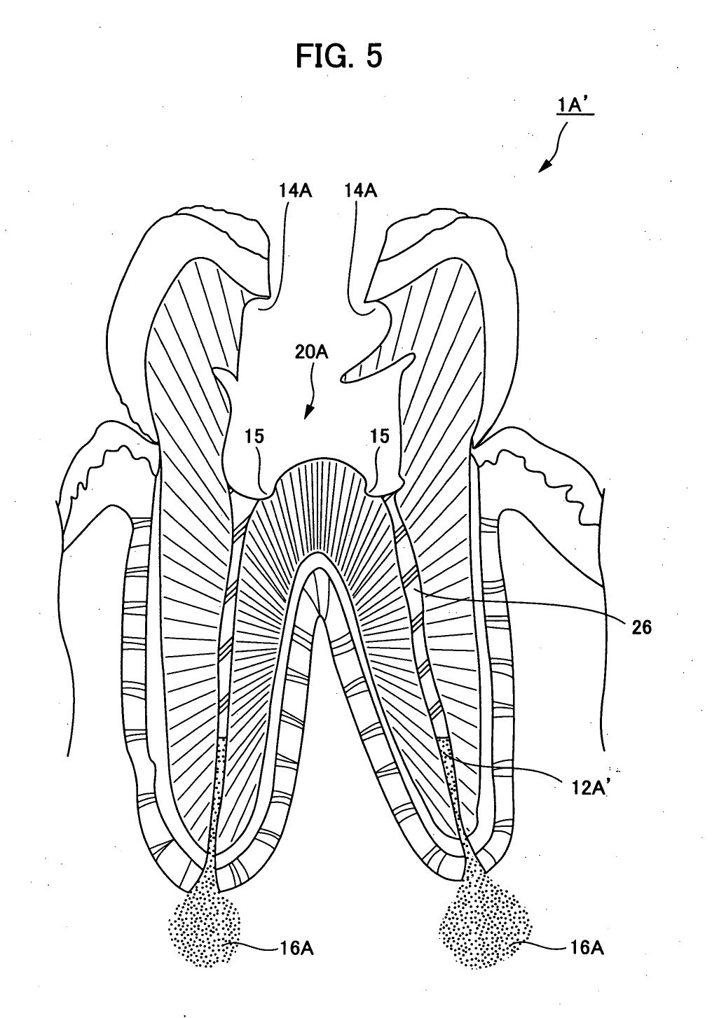

FIG. 5 depicts a cross-sectional view of the tooth 1A′ in the next stage of treating the tooth 1A of FIG. 4 with the treatment method according to the second embodiment of the present invention.

An administering site formation process of the treatment method according to the second embodiment of the present invention is a process of grinding the dentin to form the administering site 15 having a diameter larger than that of the root canal at the opening of the root canal. Depth of the administering site 15 is usually set to be not less than 2 mm.

In this way, the efficiency of delivering the treatment composition to the bacterial invasion sites such as the alveolar bone can be promoted.

### Root Canal Filling Process

The root canal filling process is, when the infected root canal treatment is performed, a process of: eliminating the necrotic dental pulp inside the root canal prior to the washing process described below and filling this root canal with the root canal filling material 26 mediating diffusion of the treatment composition into this root canal.

In this case, a complete removal of the necrotic pulp is not necessarily required, and the necrotic pulp 12A′ may remain in a deep portion of the root canal.

As a root-filling material, gutta-percha and apatite-type sealers can be used.

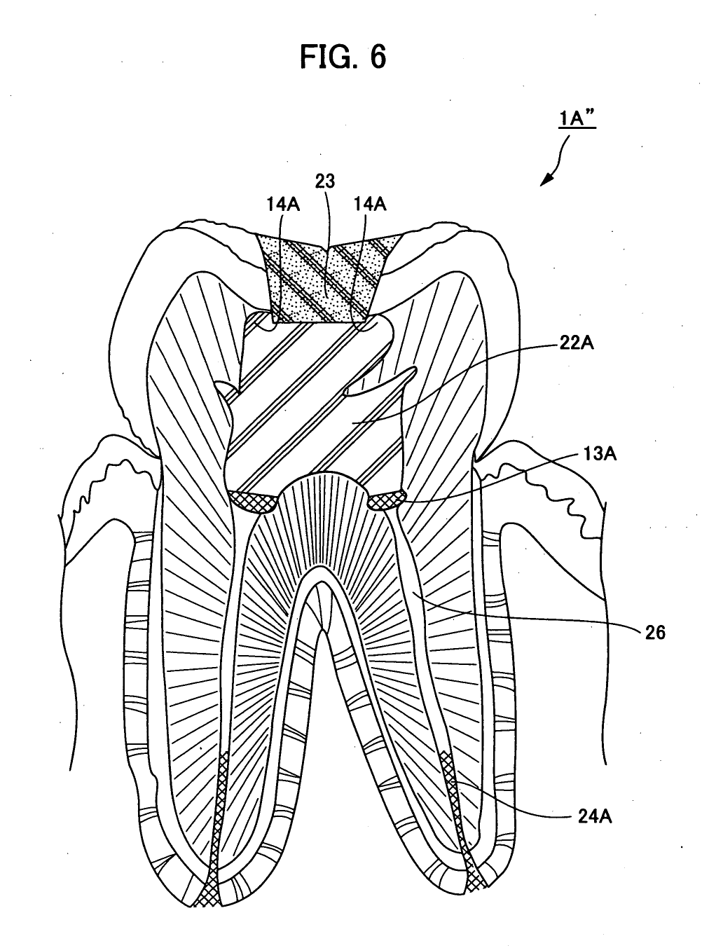

FIG. 6 depicts a cross-sectional view of the tooth 1A″ in a further stage of treating the tooth with the treatment method according to the second embodiment of the present invention.

The treatment composition diffuses from the treatment drug layer 13A through the root filling material 26 to the necrotic pulp 12A′ and to the alveolar bone such that the sterilized tissue 24A is formed and the intraoral bacteria 16A are sterilized.

With the treatment method according to the above-described embodiment, formation of the treatment drug layer 13A over the administering site 15 alone enables the diffusion of the treatment composition through dentinal canals and gaps between root canal and root-filling material to the alveolar bone, and the lime, thereby achieving the sterilization of bacterial invasion sites such as alveolar bone, and the like.

In this case, since nerves in necrotic pulps are dead, the removal thereof does not cause much pain to the subject.

With the treatment method according to the above-described embodiment, even when the root of the tooth is curved, the treatment composition passes through dentinal canals and gaps between root canal and root-filling material, diffuses and penetrates into the alveolar bone, and the like, thereby enabling the sterilization of bacterial invasion sites such as alveolar bone, etc.

### EXAMPLE

### Test Example 1

### Base

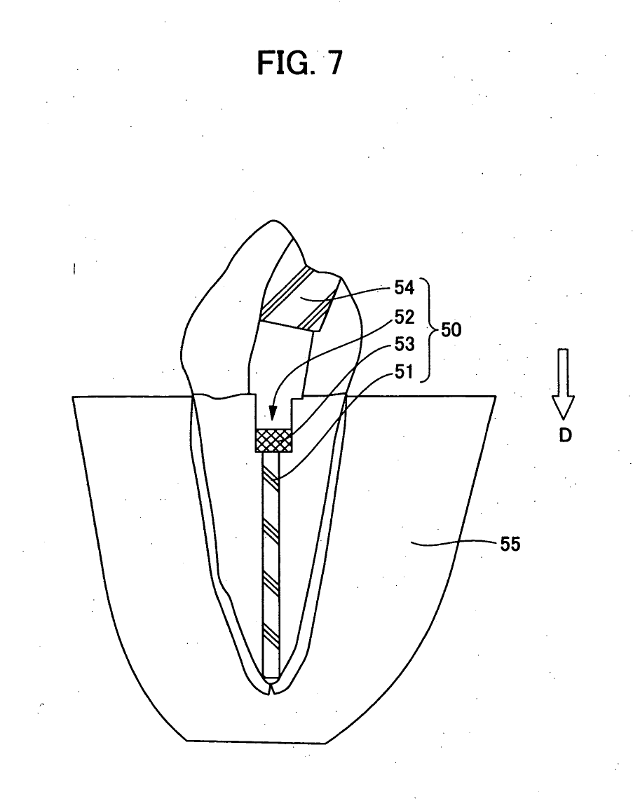

FIG. 7 depicts a cross-sectional view of a sample 50 according to the present test example.

First, the mandibular first premolar affected with apical periodontitis was drilled with a #70 reamer to enlarge the root canal and further the root canal 51 was filled using a gutta-percha point and a sealer, and by pressing them sideways. Subsequently a nearly cylindrical hole about 2 mm deep from the cervical line and about 1.5 mm in diameter was formed (hereinafter this hole is referred to as the drug application seat 52 of the above-described administering site). At the bottom of this drug application seat 52, two small pieces 53 (about 1.0 mm in diameter) of each base shown in Table 1 added with food red were loaded. Furthermore, cotton ball (not shown) was placed so as to cover these small pieces 53, and “Caviton (registered trade mark)” (G C Co., Ltd.) was layered over this cotton ball to form the covering layer 54, thereby preparing the sample 50.

After the root portion of each sample 50 was embedded in an ordinary gypsum block 55, it was stored under 100% humidity.

TABLE 1

| Sample No. | Composition |
|---|---|
| 1 | Water |
| 2 | Propylene glycol |
| 3 | “Solbase (brand name)”:propylene glycol = 1:1 |
| | (mass ratio) |
| 4 | Polyethylene glycol 4000:propylene glycol = 3:1 |
| | (mass ratio) |
| 5 | Polyethylene glycol 600 |
| 6 | Glycerin |

Migration distances of food red from the drug application seat for storing times of 24 hours and 48 hours were measured to assess the penetrability of the base contained in each sample. These results are shown in Table 2. In this case, the migration distance was determined as the longest distance in the direction of depth (in the direction of the arrow D in FIG. 7) of the area reached by coloring due to food red when the sample 50 was observed from outside.

TABLE 2

Migration distance of food red from drug
application seat (depth direction); in mm

| Sample No. | 24 hours | 48 hours |
|---|---|---|
| 1 | 2.0 | 4.0 |
| 2 | 2.0 | 3.5 |
| 3 | 5.0 | 16.0 |
| 4 | 9.5 | 18.5 |
| 5 | 3.5 | 4.0 |
| 6 | 5.0 | 7.0 |

As shown in Table 2, it was proved that penetrability of the base is best in sample #4, that is, the base containing polyethylene glycol and propylene glycol. Herein sample #4 contained polyethylene glycol 4000 and propylene glycol in a 3:1 ratio by mass, in other words, in a 1:1 volume ratio.

### Test Example 2

### Washing

In Test Example 1, prior to loading small pieces of the base (sample 2), the drug application seat was washed by each of the following washing methods.

In the washing treatment section 1, first, a cotton ball soaked in 35.2 percent volume phosphate aqueous solution was loaded onto the drug application seat, left standing for 10 seconds, washed with water, and air-blown.

In the washing treatment section 2, a cotton ball soaked in a 12 percent volume EDTA aqueous solution was loaded onto the drug application seat, left standing for 60 seconds, washed with water and air-blown.

As to each washing treatment section, using the base of sample #2 in Test Example 1, penetrability of the base was assessed by a similar method as in Test Example 1. These results are shown in Table 3.

TABLE 3

| Washing | |
|---|---|
| treatment | Migration distance of food red from drug |
| section no. | application seat (depth direction); in mm |
| 1 | 3.5 |
| 2 | 5.5 |

As shown in Table 3, since in either washing treatment sections, the migration distance of food red was increased compared to the case of Test Example 1, it was proved that washing treatment performed for the drug application seat. enables the improvement of penetrability of the base. It was demonstrated that in the washing treatment section 2, that is, when washing is performed using a solution containing 12 volume percent EDTA in particular, penetrability of the base is most improved.

Herein, the present invention is not limited to the above-described embodiments, and modifications and improvements within the scope of achieving the purpose of the present invention are included in this invention.

### INDUSTRIAL APPLICABILITY

With the present invention, the following effects can be obtained.

With a treatment method that does not include a grinding of living tissue, pain caused to the subject can be suppressed.

By administering a treatment composition containing antimicrobial agents capable of killing all intraoral bacteria and fungi to teeth, this treatment composition diffuses and penetrates in tissue that contains microbes and sterilizes them. Accordingly, since the repropagation of bacteria or fungi inside the teeth is prevented after the covering process, the recurrence of bacterial intraoral disease can be assuredly prevented. Furthermore, by the spontaneous generation of tissue repairing reactions such as calcium re-precipitation (remineralization), formation of repairing dentin, and cement propagation, the tissue thus sterilized are repaired almost to the state prior to the microbial infection, regardless of whether necrotic tissue or living tissue is involved.

Accordingly, it is possible to treat bacterial intraoral disease, and furthermore, without depending on the removal of living tissue, to sufficiently suppress pain caused to the subject and prevent the recurrence of bacterial intraoral disease.

1. A bacterial intraoral disease treatment method comprising the processes of:
washing a tooth affected by bacterial intraoral disease using a washing treatment solution,
administering a treatment composition to the washed tooth, and
covering a tooth opening, wherein the treatment composition contains:
an antibacterial agent having an antibacterial property against intraoral bacteria, and
a base containing polyethylene glycol 400, polyethylene glycol 600, polyethylene glycol 6000 and propylene glycol.

2. A treatment method according to claim 1, wherein the base contains polyethylene glycol 400 of not less than 13 percent volume and not more than 19 percent volume; polyethylene glycol 600 by not less than 13 percent volume and not more than 19 percent volume; polyethylene glycol 6000 by not less than 27 percent volume and not more than 38 percent volume; and propylene glycol not less than 36 percent volume and not more than 50 percent volume.

3. A treatment method according to claim 1, wherein the washing treatment solution contains EDTA at a pH of about 7 and a water-soluble thickener containing no metallic ions.

4. A treatment method according to claim 3, wherein the washing treatment solution contains EDTA of not less than 10 percent volume and not more than 12 percent volume of this washing treatment solution.

5. A treatment method according to claim 1, further comprising a hemostatic process of stopping bleeding from pulp and/or gingival prior to the covering process, wherein the hemostatic process is a process of stopping the bleeding from pulp and/or gingival using a hemostatic treatment solution containing sodium alginate and zinc oxide.

6. A treatment method according to claim 5, wherein the hemostatic treatment solution contains sodium alginate of not less than 6.0 percent volume and not more than 6.5 percent volume of this hemostatic treatment solution, and zinc oxide of not less than 33 percent volume and not more than 35 percent volume.

7. A treatment composition for bacterial intraoral disease comprising an antibacterial agent having an antibacterial property against intraoral bacteria and a base containing polyethylene glycol 400, polyethylene glycol 600, polyethylene glycol 6000, and propylene glycol.

8. A treatment composition according to claim 7, wherein the base contains polyethylene glycol 400 of not less than 13 percent volume and not more than 19 percent volume of this base; polyethylene glycol 600 of not less than 13 percent volume and not more than 19 percent volume; polyethylene glycol 6000 of not less than 27 percent volume and not more than 38 percent volume; and propylene glycol of not less than 36 percent volume and not more than 50 percent volume.

9. A washing treatment solution to be used for washing teeth comprising EDTA at a pH of about 7 and a water-soluble thickener containing no metallic ions.

10. A washing treatment solution according to claim 9 containing EDTA of not less than 10 percent volume and not more than 12 percent volume.

11. A hemostatic treatment solution used for stopping bleeding from pulp and/or gingival comprising sodium alginate and zinc oxide.

12. A hemostatic treatment solution according to claim 11 comprising sodium alginate of not less than 6.0 percent volume and not more than 6.5 percent volume as well as zinc oxide of not less than 33 percent volume and not more than 35 percent volume.

13. A treatment kit for bacterial intraoral diseases comprising:
basic ingredients of a treatment composition for bacterial intraoral disease comprising an antibacterial agent having an antibacterial property against intraoral bacteria and a base containing polyethylene glycol 400, polyethylene glycol 600, polyethylene glycol 6000, and propylene glycol;
a washing treatment solution to be used for washing teeth comprising EDTA at a pH of about 7 and a water-soluble thickener containing no metallic ions; and
a hemostatic treatment solution used for stopping bleeding from pulp and/or gingival comprising sodium alginate and zinc oxide.

14. A treatment method according to claim 2, further comprising a hemostatic process of stopping bleeding from pulp and/or gingival prior to the covering process, wherein the hemostatic process is a process of stopping the bleeding from pulp and/or gingival using a hemostatic treatment solution containing sodium alginate and zinc oxide.

15. A treatment method according to claim 3, further comprising a hemostatic process of stopping bleeding from pulp and/or gingival prior to the covering process, wherein the hemostatic process is a process of stopping the bleeding from pulp and/or gingival using a hemostatic treatment solution containing sodium alginate and zinc oxide.

16. A treatment method according to claim 4, further comprising a hemostatic process of stopping bleeding from pulp and/or gingival prior to the covering process, wherein the hemostatic process is a process of stopping the bleeding from pulp and/or gingival using a hemostatic treatment solution containing sodium alginate and zinc oxide.
